(19) United States
(12) Patent Application Publication
(10) Pub. No.: US 2026/0297456 A1
(43) Pub. Date: Oct. 1, 2026

(54) Wash-active compounds based on a combination of anions and cationic detergent

(71) Applicant: Henkel AG & Co. KGaA, Dusseldorf (DE)

(72) Inventors: Caroline Hartwig, Essen (DE); Peter Schmiedel, Dusseldorf (DE); Mareile Job, Leverkusen (DE); Mahdis Hesami, Dusseldorf (DE)

(21) Appl. No.: 18/881,354

(22) PCT Filed: Mar. 30, 2023
(86) PCT No.: PCT/EP2023/058263

§ 371 Date: Jan. 6, 2025

(30) Foreign Application Priority Data
Jul. 13, 2022 (DE) 10 2022 207 153.0

(51) Int. Cl. C11D 1/28; C11D 1/66; C11D 3/20; C11D 3/36; C11D 3/37; C11D 3/386; C11D 3/43

(52) U.S. Cl. CPC C11D 1/28; C11D 1/66; C11D 3/2079; C11D 3/361; C11D 3/37; C11D 3/386; C11D 3/43; C11D 2111/12

(57) ABSTRACT

The invention relates to a compound of the general formula (1) wherein i) R is selected from the group of the C₆ to C₂₀ alkyl groups; ii) X is selected from the group a) C₆H₄, b) (OC₂H₄)ₙO with n=1 to 8, c) C(O)—N(CH₃)CH₂CH₂, d) O(O)CCH₂(RO(O)C)CH with R=C₆ to C₂₀ alkyl, iii) R₂ selected from the group of the C₆ to C₂₀ alkyl groups, iv) with m+n=5 to 12, to detergents or cleaning agents that contain this compound, and to processes for preparing this compound.

The present invention relates to a wash-active compound with increased washing performance in the case of greasy stains. The application furthermore relates to detergents and cleaning agents that contain this wash-active compound, to processes for preparing the wash-active compound, and to detergents and cleaning processes using the wash-active compound.

Continuously changing requirements are placed on the forms of manufacture and supply of washing and cleaning agents. A trend relevant to the preparation of detergents and cleaning agents is their concentration. In addition to higher consumer acceptance due to simplified handling, the background of this development is, in particular, sustainability aspects, for example in relation to transport volumes and costs and the quantity of packaging materials used.

In concentrated detergents and cleaning agents, the anionic detergent acids are usually present in a form neutralized by organic alkanolamines, such as monoethanolamine (MEA), monoisopropanolamine (MIPA) or triethanolamine (TEA). However, the ammonium ions resulting from the neutralization reaction do not contribute to washing performance.

Against this background, the application was based on the object of providing wash- and clean-active substances that enable the preparation of visually appealing, concentrated flowable detergent preparations. The active ingredients should be producible in a simple and efficient manner, have good storage capacity, and be characterized in particular by good cleaning results, for example on greasy stains.

Surprisingly, it was found that the neutralization products of anionic detergent acids with fatty amine ethoxylates of suitable ethylene oxide chain lengths have wash-active compounds with a surprising high washing performance on greasy stains. In addition, it was found that the washing performance of detergent compositions that contain these neutralization products does not necessarily require the addition of further detergents, for example non-ionic detergents due to their good washing performance.

The international patent application WO 2017/205915 A1 describes an aqueous cleaning agent composition that contains silicates, ethoxylated alcohols, quaternary fatty amine ethoxylates, and a urea salt.

In the European patent EP 0 231 886 B1, fatty amine ethoxylates are described as solubilizers in cleaning concentrates for cleaning agent solutions for bottle cleaning.

A first subject of this application is a compound of general formula (1)

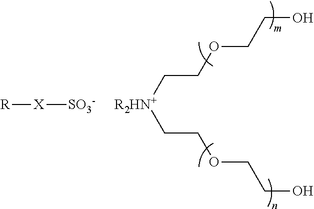

i) R is selected from the group of C₆ to C₂₀ alkyl groups;
ii) X is selected from the group
   a) C₆H₄
   b) (OC₂H₄)ₙO where n=1 to 8
   c) C(O)—N(CH₃)CH₂CH₂
   d) O(O)CCH₂(RO(O)C)CH where R=C₆ to C₂₀ alkyl
iii) R₂ is selected from the group of C₆ to C₂₀ alkyl groups
iv) where m+n=5 to 12.

Surprisingly, it was found that by the neutralization of specific anionic detergent acids containing sulfonic acid groups with the above-mentioned fatty amine ethoxylates, surface-active substances can be obtained, which are suitable, for example, for the preparation of solvent-free liquid detergents with active substances of up to 100 wt. %. Furthermore, it was found that the neutralization products of sulfonic acid group-containing anionic detergent acids with the above-mentioned fatty amine ethoxylates are characterized by an increased reduction in the interfacial tension of water and improved fat cleaning compared to conventional wash-active compounds, for example the neutralization products of sulfonic acid group-containing anionic detergent acids with alkanolamines.

A particularly preferred compound of general formula (1) is based on anionic detergents from the group of alkylbenzenesulfonic acids. Particularly preferred compounds of general formula (1) are therefore characterized in that
i) R is selected from the group of C₁₀ to C₁₄ alkyl groups;
ii) X is selected from group C₆H₄
iii) R₂ is selected from the group of C₆ to C₂₀ alkyl groups, preferably C₁₀ to C₁₆ alkyl groups
iv) where m+n=5 to 12.

Groups R selected from the group of linear C₁₀ to C₁₄ alkyl groups, of secondary C₁₀ to C₁₄ alkyl groups and in particular the linear secondary C₁₀ to C₁₄ alkyl groups have proven to be particularly advantageous. Very particular preference is given to groups R from the group of linear C₁₂H₂₅ alkyl groups, in particular the linear secondary C₁₂H₂₅ alkyl groups.

In alternative embodiments, the sulfonic acid on which the compound of general formula (1) is based can be varied.

A first alternative forms compounds of general formula (1), wherein
i) R is selected from the group of C₁₂ to C₂₀ alkyl groups;
ii) X is selected from the group selected from the group (OC₂H₄)ₙO where n=1 to 8
iii) R₂ is selected from the group of C₆ to C₂₀ alkyl groups
iv) where m+n=5 to 12

A further alternative comprises compounds of general formula (1), wherein
i) R is selected from the group of C₆ to C₂₀ alkyl groups;
ii) X is selected from the group C(O)—N(CH₃)CH₂CH₂;
iii) R₂ is selected from the group of C₆ to C₂₀ alkyl groups
iv) where m+n=5 to 12

The third alternative represents compounds of general formula (1), wherein
i) R is selected from the group of C₇ to C₁₇ alkyl groups;
ii) X is selected from the group O(O)CCH₂(RO(O)C)CH where R=C₆ to C₁₇ alkyl;
iii) R₂ is selected from the group of C₆ to C₂₀ alkyl groups
iv) where m+n=5 to 12

In addition to the sulfonic acid group-containing anionic detergent acids, the compounds of general formula (1) furthermore comprise specific fatty amine ethoxylates. The ethoxylated side chains characterizing the fatty amine ethoxylates can have a degree of ethoxylation of m+n=5 to 12. For example, the degree of ethoxylation can be m+n=5, or m+n=6, or m+n=7, or m+n=8, or m+n=9, or m+n=10, or m+n=11, or m+n=12. Particular preference is given to compounds of general formula (1) where m+n=5 or 6.

The degree of ethoxylation of the two ethoxylated side chains can be the same or different. Because they are easier to produce, fatty amine ethoxylates with the same degree of ethoxylation of the side chains are preferred.

In a particularly preferred compound of general formula (1)
i) R represents a linear secondary C₁₂H₂₅ alkyl group;
ii) X is selected from group C₆H₄
iii) R₂ is selected from the group of C₁₂H₂₅ alkyl groups
iv) m+n=5 or 6

In particular, the neutralization products of alkylbenzenesulfonic acid-based anionic detergent acids with the above-mentioned fatty amine ethoxylates are characterized by a greater reduction of the interfacial tension of water and improved fat cleaning compared to conventional wash-active compounds, for example the neutralization products of alkylbenzenesulfonic acid-based anionic detergent acids with alkanolamines.

Some preferred compounds of general formula (1) where
i) R is selected from the group of C₆ to C₂₀ alkyl groups;
ii) X is selected from the group
   a) C₆H₄
   b) (OC₂H₄)ₙO where n=1 to 8
   c) C(O)—N(CH₃)CH₂CH₂
   d) O(O)CCH₂(RO(O)C)CH where R=C₆ to C₂₀ alkyl
iii) R₂ is selected from the group of C₆ to C₂₀ alkyl groups
iv) where m+n=5 to 12.
   can be found in the following tables:

TABLE a

| | R | X | R₂ | m + n |
|---|---|---|---|---|
| 1a | C₆ to C₂₀ alkyl | C₆H₄ | C₆ to C₂₀ alkyl | 5 to 12 |
| 2a | C₆ to C₂₀ alkyl | C₆H₄ | C₆ to C₂₀ alkyl | 5 |
| 3a | C₆ to C₂₀ alkyl | C₆H₄ | C₆ to C₂₀ alkyl | 6 |
| 4a | C₆ to C₂₀ alkyl | C₆H₄ | C₆ to C₂₀ alkyl | 7 |
| 5a | C₆ to C₂₀ alkyl | C₆H₄ | C₆ to C₂₀ alkyl | 8 |
| 6a | C₆ to C₂₀ alkyl | C₆H₄ | C₆ to C₂₀ alkyl | 9 |
| 7a | C₆ to C₂₀ alkyl | C₆H₄ | C₆ to C₂₀ alkyl | 10 |
| 8a | C₆ to C₂₀ alkyl | C₆H₄ | C₆ to C₂₀ alkyl | 11 |
| 9a | C₆ to C₂₀ alkyl | C₆H₄ | C₆ to C₂₀ alkyl | 12 |
| 10a | C₁₀ to C₁₄ alkyl | C₆H₄ | C₆ to C₂₀ alkyl | 5 to 12 |
| 11a | C₁₀ to C₁₄ alkyl | C₆H₄ | C₆ to C₂₀ alkyl | 5 |
| 12a | C₁₀ to C₁₄ alkyl | C₆H₄ | C₆ to C₂₀ alkyl | 6 |
| 13a | C₁₀ to C₁₄ alkyl | C₆H₄ | C₆ to C₂₀ alkyl | 7 |
| 14a | C₁₀ to C₁₄ alkyl | C₆H₄ | C₆ to C₂₀ alkyl | 8 |
| 15a | C₁₀ to C₁₄ alkyl | C₆H₄ | C₆ to C₂₀ alkyl | 9 |
| 16a | C₁₀ to C₁₄ alkyl | C₆H₄ | C₆ to C₂₀ alkyl | 10 |
| 17a | C₁₀ to C₁₄ alkyl | C₆H₄ | C₆ to C₂₀ alkyl | 11 |
| 18a | C₁₀ to C₁₄ alkyl | C₆H₄ | C₆ to C₂₀ alkyl | 12 |
| 19a | C₆ to C₂₀ alkyl | C₆H₄ | C₁₀ to C₁₆ alkyl | 5 to 12 |
| 20a | C₆ to C₂₀ alkyl | C₆H₄ | C₁₀ to C₁₆ alkyl | 5 |
| 21a | C₆ to C₂₀ alkyl | C₆H₄ | C₁₀ to C₁₆ alkyl | 6 |
| 22a | C₆ to C₂₀ alkyl | C₆H₄ | C₁₀ to C₁₆ alkyl | 7 |
| 23a | C₆ to C₂₀ alkyl | C₆H₄ | C₁₀ to C₁₆ alkyl | 8 |
| 24a | C₆ to C₂₀ alkyl | C₆H₄ | C₁₀ to C₁₆ alkyl | 9 |
| 25a | C₆ to C₂₀ alkyl | C₆H₄ | C₁₀ to C₁₆ alkyl | 10 |
| 26a | C₆ to C₂₀ alkyl | C₆H₄ | C₁₀ to C₁₆ alkyl | 11 |
| 27a | C₆ to C₂₀ alkyl | C₆H₄ | C₁₀ to C₁₆ alkyl | 12 |
| 28a | C₁₂H₂₅ alkyl | C₆H₄ | C₆ to C₂₀ alkyl | 5 to 12 |
| 29a | C₁₂H₂₅ alkyl | C₆H₄ | C₆ to C₂₀ alkyl | 5 |
| 30a | C₁₂H₂₅ alkyl | C₆H₄ | C₆ to C₂₀ alkyl | 6 |
| 31a | C₁₂H₂₅ alkyl | C₆H₄ | C₆ to C₂₀ alkyl | 7 |
| 32a | C₁₂H₂₅ alkyl | C₆H₄ | C₆ to C₂₀ alkyl | 8 |
| 33a | C₁₂H₂₅ alkyl | C₆H₄ | C₆ to C₂₀ alkyl | 9 |
| 34a | C₁₂H₂₅ alkyl | C₆H₄ | C₆ to C₂₀ alkyl | 10 |
| 35a | C₁₂H₂₅ alkyl | C₆H₄ | C₆ to C₂₀ alkyl | 11 |
| 36a | C₁₂H₂₅ alkyl | C₆H₄ | C₆ to C₂₀ alkyl | 12 |

TABLE b

| | R | X | R₂ | m + n |
|---|---|---|---|---|
| 1b | C₆ to C₂₀ | (OC₂H₄)ₙ O where | C₆ to C₂₀ | 5 to 12 |
| | alkyl | n = 1 to 8 | alkyl | |
| 2b | C₆ to C₂₀ | (OC₂H₄)ₙ O where | C₆ to C₂₀ | 5 |
| | alkyl | n = 1 to 8 | alkyl | |
| 3b | C₆ to C₂₀ | (OC₂H₄)ₙ O where | C₆ to C₂₀ | 6 |
| | alkyl | n = 1 to 8 | alkyl | |
| 4b | C₆ to C₂₀ | (OC₂H₄)ₙ O where | C₆ to C₂₀ | 7 |
| | alkyl | n = 1 to 8 | alkyl | |
| 5b | C₆ to C₂₀ | (OC₂H₄)ₙ O where | C₆ to C₂₀ | 8 |
| | alkyl | n = 1 to 8 | alkyl | |
| 6b | C₆ to C₂₀ | (OC₂H₄)ₙ O where | C₆ to C₂₀ | 9 |
| | alkyl | n = 1 to 8 | alkyl | |
| 7b | C₆ to C₂₀ | (OC₂H₄)ₙ O where | C₆ to C₂₀ | 10 |
| | alkyl | n = 1 to 8 | alkyl | |
| 8b | C₆ to C₂₀ | (OC₂H₄)ₙ O where | C₆ to C₂₀ | 11 |
| | alkyl | n = 1 to 8 | alkyl | |
| 9b | C₆ to C₂₀ | (OC₂H₄)ₙ O where | C₆ to C₂₀ | 12 |
| | alkyl | n = 1 to 8 | alkyl | |
| 10b | C₁₀ to C₁₄ | (OC₂H₄)ₙ O where | C₆ to C₂₀ | 5 to 12 |
| | alkyl | n = 1 to 8 | alkyl | |
| 11b | C₁₀ to C₁₄ | (OC₂H₄)ₙ O where | C₆ to C₂₀ | 5 |
| | alkyl | n = 1 to 8 | alkyl | |
| 12b | C₁₀ to C₁₄ | (OC₂H₄)ₙ O where | C₆ to C₂₀ | 6 |
| | alkyl | n = 1 to 8 | alkyl | |
| 13b | C₁₀ to C₁₄ | (OC₂H₄)ₙ O where | C₆ to C₂₀ | 7 |
| | alkyl | n = 1 to 8 | alkyl | |
| 14b | C₁₀ to C₁₄ | (OC₂H₄)ₙ O where | C₆ to C₂₀ | 8 |
| | alkyl | n = 1 to 8 | alkyl | |
| 15b | C₁₀ to C₁₄ | (OC₂H₄)ₙ O where | C₆ to C₂₀ | 9 |
| | alkyl | n = 1 to 8 | alkyl | |
| 16b | C₁₀ to C₁₄ | (OC₂H₄)ₙ O where | C₆ to C₂₀ | 10 |
| | alkyl | n = 1 to 8 | alkyl | |
| 17b | C₁₀ to C₁₄ | (OC₂H₄)ₙ O where | C₆ to C₂₀ | 11 |
| | alkyl | n = 1 to 8 | alkyl | |
| 18b | C₁₀ to C₁₄ | (OC₂H₄)ₙ O where | C₆ to C₂₀ | 12 |
| | alkyl | n = 1 to 8 | alkyl | |
| 19b | C₆ to C₂₀ | (OC₂H₄)ₙ O where | C₁₀ to C₁₆ | 5 to 12 |
| | alkyl | n = 1 to 8 | alkyl | |
| 20b | C₆ to C₂₀ | (OC₂H₄)ₙ O where | C₁₀ to C₁₆ | 5 |
| | alkyl | n = 1 to 8 | alkyl | |
| 21b | C₆ to C₂₀ | (OC₂H₄)ₙ O where | C₁₀ to C₁₆ | 6 |
| | alkyl | n = 1 to 8 | alkyl | |
| 22b | C₆ to C₂₀ | (OC₂H₄)ₙ O where | C₁₀ to C₁₆ | 7 |
| | alkyl | n = 1 to 8 | alkyl | |
| 23b | C₆ to C₂₀ | (OC₂H₄)ₙ O where | C₁₀ to C₁₆ | 8 |
| | alkyl | n = 1 to 8 | alkyl | |
| 24b | C₆ to C₂₀ | (OC₂H₄)ₙ O where | C₁₀ to C₁₆ | 9 |
| | alkyl | n = 1 to 8 | alkyl | |
| 25b | C₆ to C₂₀ | (OC₂H₄)ₙ O where | C₁₀ to C₁₆ | 10 |
| | alkyl | n = 1 to 8 | alkyl | |
| 26b | C₆ to C₂₀ | (OC₂H₄)ₙ O where | C₁₀ to C₁₆ | 11 |
| | alkyl | n = 1 to 8 | alkyl | |
| 27b | C₆ to C₂₀ | (OC₂H₄)ₙ O where | C₁₀ to C₁₆ | 12 |
| | alkyl | n = 1 to 8 | alkyl | |
| 28b | C₁₂H₂₅ | (OC₂H₄)ₙ O where | C₆ to C₂₀ | 5 to 12 |
| | alkyl | n = 1 to 8 | alkyl | |
| 29b | C₁₂H₂₅ | (OC₂H₄)ₙ O where | C₆ to C₂₀ | 5 |
| | alkyl | n = 1 to 8 | alkyl | |
| 30b | C₁₂H₂₅ | (OC₂H₄)ₙ O where | C₆ to C₂₀ | 6 |
| | alkyl | n = 1 to 8 | alkyl | |
| 31b | C₁₂H₂₅ | (OC₂H₄)ₙ O where | C₆ to C₂₀ | 7 |
| | alkyl | n = 1 to 8 | alkyl | |
| 32b | C₁₂H₂₅ | (OC₂H₄)ₙ O where | C₆ to C₂₀ | 8 |
| | alkyl | n = 1 to 8 | alkyl | |
| 33b | C₁₂H₂₅ | (OC₂H₄)ₙ O where | C₆ to C₂₀ | 9 |
| | alkyl | n = 1 to 8 | alkyl | |
| 34b | C₁₂H₂₅ | (OC₂H₄)ₙ O where | C₆ to C₂₀ | 10 |
| | alkyl | n = 1 to 8 | alkyl | |
| 35b | C₁₂H₂₅ | (OC₂H₄)ₙ O where | C₆ to C₂₀ | 11 |
| | alkyl | n = 1 to 8 | alkyl | |
| 36b | C₁₂H₂₅ | (OC₂H₄)ₙ O where | C₆ to C₂₀ | 12 |
| | alkyl | n = 1 to 8 | alkyl | |

TABLE c

| | R | X | R₂ | m + n |
|---|---|---|---|---|
| 1c | C₆ to C₂₀ | C(O)—N(CH₃)CH₂CH₂ | C₆ to C₂₀ alkyl | 5 to 12 |
| | alkyl | | | |
| 2c | C₆ to C₂₀ | C(O)—N(CH₃)CH₂CH₂ | C₆ to C₂₀ alkyl | 5 |
| | alkyl | | | |
| 3c | C₆ to C₂₀ | C(O)—N(CH₃)CH₂CH₂ | C₆ to C₂₀ alkyl | 6 |
| | alkyl | | | |
| 4c | C₆ to C₂₀ | C(O)—N(CH₃)CH₂CH₂ | C₆ to C₂₀ alkyl | 7 |
| | alkyl | | | |
| 5c | C₆ to C₂₀ | C(O)—N(CH₃)CH₂CH₂ | C₆ to C₂₀ alkyl | 8 |
| | alkyl | | | |
| 6c | C₆ to C₂₀ | C(O)—N(CH₃)CH₂CH₂ | C₆ to C₂₀ alkyl | 9 |
| | alkyl | | | |
| 7c | C₆ to C₂₀ | C(O)—N(CH₃)CH₂CH₂ | C₆ to C₂₀ alkyl | 10 |
| | alkyl | | | |
| 8c | C₆ to C₂₀ | C(O)—N(CH₃)CH₂CH₂ | C₆ to C₂₀ alkyl | 11 |
| | alkyl | | | |
| 9c | C₆ to C₂₀ | C(O)—N(CH₃)CH₂CH₂ | C₆ to C₂₀ alkyl | 12 |
| | alkyl | | | |
| 10c | C₁₀ to C₁₄ | C(O)—N(CH₃)CH₂CH₂ | C₆ to C₂₀ alkyl | 5 to 12 |
| | alkyl | | | |
| 11c | C₁₀ to C₁₄ | C(O)—N(CH₃)CH₂CH₂ | C₆ to C₂₀ alkyl | 5 |
| | alkyl | | | |
| 12c | C₁₀ to C₁₄ | C(O)—N(CH₃)CH₂CH₂ | C₆ to C₂₀ alkyl | 6 |
| | alkyl | | | |
| 13c | C₁₀ to C₁₄ | C(O)—N(CH₃)CH₂CH₂ | C₆ to C₂₀ alkyl | 7 |
| | alkyl | | | |
| 14c | C₁₀ to C₁₄ | C(O)—N(CH₃)CH₂CH₂ | C₆ to C₂₀ alkyl | 8 |
| | alkyl | | | |
| 15c | C₁₀ to C₁₄ | C(O)—N(CH₃)CH₂CH₂ | C₆ to C₂₀ alkyl | 9 |
| | alkyl | | | |
| 16c | C₁₀ to C₁₄ | C(O)—N(CH₃)CH₂CH₂ | C₆ to C₂₀ alkyl | 10 |
| | alkyl | | | |
| 17c | C₁₀ to C₁₄ | C(O)—N(CH₃)CH₂CH₂ | C₆ to C₂₀ alkyl | 11 |
| | alkyl | | | |
| 18c | C₁₀ to C₁₄ | C(O)—N(CH₃)CH₂CH₂ | C₆ to C₂₀ alkyl | 12 |
| | alkyl | | | |
| 19c | C₆ to C₂₀ | C(O)—N(CH₃)CH₂CH₂ | C₁₀ to C₁₆ alkyl | 5 to 12 |
| | alkyl | | | |
| 20c | C₆ to C₂₀ | C(O)—N(CH₃)CH₂CH₂ | C₁₀ to C₁₆ alkyl | 5 |
| | alkyl | | | |
| 21c | C₆ to C₂₀ | C(O)—N(CH₃)CH₂CH₂ | C₁₀ to C₁₆ alkyl | 6 |
| | alkyl | | | |
| 22c | C₆ to C₂₀ | C(O)—N(CH₃)CH₂CH₂ | C₁₀ to C₁₆ alkyl | 7 |
| | alkyl | | | |
| 23c | C₆ to C₂₀ | C(O)—N(CH₃)CH₂CH₂ | C₁₀ to C₁₆ alkyl | 8 |
| | alkyl | | | |
| 24c | C₆ to C₂₀ | C(O)—N(CH₃)CH₂CH₂ | C₁₀ to C₁₆ alkyl | 9 |
| | alkyl | | | |
| 25c | C₆ to C₂₀ | C(O)—N(CH₃)CH₂CH₂ | C₁₀ to C₁₆ alkyl | 10 |
| | alkyl | | | |
| 26c | C₆ to C₂₀ | C(O)—N(CH₃)CH₂CH₂ | C₁₀ to C₁₆ alkyl | 11 |
| | alkyl | | | |
| 27c | C₆ to C₂₀ | C(O)—N(CH₃)CH₂CH₂ | C₁₀ to C₁₆ alkyl | 12 |
| | alkyl | | | |
| 28c | C₁₂H₂₅ alkyl | C(O)—N(CH₃)CH₂CH₂ | C₆ to C₂₀ alkyl | 5 to 12 |
| 29c | C₁₂H₂₅ alkyl | C(O)—N(CH₃)CH₂CH₂ | C₆ to C₂₀ alkyl | 5 |
| 30c | C₁₂H₂₅ alkyl | C(O)—N(CH₃)CH₂CH₂ | C₆ to C₂₀ alkyl | 6 |
| 31c | C₁₂H₂₅ alkyl | C(O)—N(CH₃)CH₂CH₂ | C₆ to C₂₀ alkyl | 7 |
| 32c | C₁₂H₂₅ alkyl | C(O)—N(CH₃)CH₂CH₂ | C₆ to C₂₀ alkyl | 8 |
| 33c | C₁₂H₂₅ alkyl | C(O)—N(CH₃)CH₂CH₂ | C₆ to C₂₀ alkyl | 9 |
| 34c | C₁₂H₂₅ alkyl | C(O)—N(CH₃)CH₂CH₂ | C₆ to C₂₀ alkyl | 10 |
| 35c | C₁₂H₂₅ alkyl | C(O)—N(CH₃)CH₂CH₂ | C₆ to C₂₀ alkyl | 11 |
| 36c | C₁₂H₂₅ alkyl | C(O)—N(CH₃)CH₂CH₂ | C₆ to C₂₀ alkyl | 12 |

TABLE d

| | R | X | R₂ | m + n |
|---|---|---|---|---|
| 1d | C₆ to C₂₀ | O(O)CCH₂(RO(O)C)CH where | C₆ to C₂₀ | 5 to 12 |
| | alkyl | R = C₆ to C₂₀ alkyl | alkyl | |
| 2d | C₆ to C₂₀ | O(O)CCH₂(RO(O)C)CH where | C₆ to C₂₀ | 5 |
| | alkyl | R = C₆ to C₂₀ alkyl | alkyl | |
| 3d | C₆ to C₂₀ | O(O)CCH₂(RO(O)C)CH where | C₆ to C₂₀ | 6 |
| | alkyl | R = C₆ to C₂₀ alkyl | alkyl | |
| 4d | C₆ to C₂₀ | O(O)CCH₂(RO(O)C)CH where | C₆ to C₂₀ | 7 |
| | alkyl | R = C₆ to C₂₀ alkyl | alkyl | |
| 5d | C₆ to C₂₀ | O(O)CCH₂(RO(O)C)CH where | C₆ to C₂₀ | 8 |
| | alkyl | R = C₆ to C₂₀ alkyl | alkyl | |
| 6d | C₆ to C₂₀ | O(O)CCH₂(RO(O)C)CH where | C₆ to C₂₀ | 9 |
| | alkyl | R = C₆ to C₂₀ alkyl | alkyl | |
| 7d | C₆ to C₂₀ | O(O)CCH₂(RO(O)C)CH where | C₆ to C₂₀ | 10 |
| | alkyl | R = C₆ to C₂₀ alkyl | alkyl | |
| 8d | C₆ to C₂₀ | O(O)CCH₂(RO(O)C)CH where | C₆ to C₂₀ | 11 |
| | alkyl | R = C₆ to C₂₀ alkyl | alkyl | |
| 9d | C₆ to C₂₀ | O(O)CCH₂(RO(O)C)CH where | C₆ to C₂₀ | 12 |
| | alkyl | R = C₆ to C₂₀ alkyl | alkyl | |
| 10d | C₁₀ to C₁₄ | O(O)CCH₂(RO(O)C)CH where | C₆ to C₂₀ | 5 to 12 |
| | alkyl | R = C₆ to C₂₀ alkyl | alkyl | |
| 11d | C₁₀ to C₁₄ | O(O)CCH₂(RO(O)C)CH where | C₆ to C₂₀ | 5 |
| | alkyl | R = C₆ to C₂₀ alkyl | alkyl | |
| 12d | C₁₀ to C₁₄ | O(O)CCH₂(RO(O)C)CH where | C₆ to C₂₀ | 6 |
| | alkyl | R = C₆ to C₂₀ alkyl | alkyl | |
| 13d | C₁₀ to C₁₄ | O(O)CCH₂(RO(O)C)CH where | C₆ to C₂₀ | 7 |
| | alkyl | R = C₆ to C₂₀ alkyl | alkyl | |
| 14d | C₁₀ to C₁₄ | O(O)CCH₂(RO(O)C)CH where | C₆ to C₂₀ | 8 |
| | alkyl | R = C₆ to C₂₀ alkyl | alkyl | |
| 15d | C₁₀ to C₁₄ | O(O)CCH₂(RO(O)C)CH where | C₆ to C₂₀ | 9 |
| | alkyl | R = C₆ to C₂₀ alkyl | alkyl | |
| 16d | C₁₀ to C₁₄ | O(O)CCH₂(RO(O)C)CH where | C₆ to C₂₀ | 10 |
| | alkyl | R = C₆ to C₂₀ alkyl | alkyl | |
| 17d | C₁₀ to C₁₄ | O(O)CCH₂(RO(O)C)CH where | C₆ to C₂₀ | 11 |
| | alkyl | R = C₆ to C₂₀ alkyl | alkyl | |
| 18d | C₁₀ to C₁₄ | O(O)CCH₂(RO(O)C)CH where | C₆ to C₂₀ | 12 |
| | alkyl | R = C₆ to C₂₀ alkyl | alkyl | |
| 19d | C₆ to C₂₀ | O(O)CCH₂(RO(O)C)CH where | C₁₀ to C₁₆ | 5 to 12 |
| | alkyl | R = C₆ to C₂₀ alkyl | alkyl | |
| 20d | C₆ to C₂₀ | O(O)CCH₂(RO(O)C)CH where | C₁₀ to C₁₆ | 5 |
| | alkyl | R = C₆ to C₂₀ alkyl | alkyl | |
| 21d | C₆ to C₂₀ | O(O)CCH₂(RO(O)C)CH where | C₁₀ to C₁₆ | 6 |
| | alkyl | R = C₆ to C₂₀ alkyl | alkyl | |
| 22d | C₆ to C₂₀ | O(O)CCH₂(RO(O)C)CH where | C₁₀ to C₁₆ | 7 |
| | alkyl | R = C₆ to C₂₀ alkyl | alkyl | |
| 23d | C₆ to C₂₀ | O(O)CCH₂(RO(O)C)CH where | C₁₀ to C₁₆ | 8 |
| | alkyl | R = C₆ to C₂₀ alkyl | alkyl | |
| 24d | C₆ to C₂₀ | O(O)CCH₂(RO(O)C)CH where | C₁₀ to C₁₆ | 9 |
| | alkyl | R = C₆ to C₂₀ alkyl | alkyl | |
| 25d | C₆ to C₂₀ | O(O)CCH₂(RO(O)C)CH where | C₁₀ to C₁₆ | 10 |
| | alkyl | R = C₆ to C₂₀ alkyl | alkyl | |
| 26d | C₆ to C₂₀ | O(O)CCH₂(RO(O)C)CH where | C₁₀ to C₁₆ | 11 |
| | alkyl | R = C₆ to C₂₀ alkyl | alkyl | |
| 27d | C₆ to C₂₀ | O(O)CCH₂(RO(O)C)CH where | C₁₀ to C₁₆ | 12 |
| | alkyl | R = C₆ to C₂₀ alkyl | alkyl | |
| 28d | C₁₂H₂₅ | O(O)CCH₂(RO(O)C)CH where | C₆ to C₂₀ | 5 to 12 |
| | alkyl | R = C₆ to C₂₀ alkyl | alkyl | |
| 29d | C₁₂H₂₅ | O(O)CCH₂(RO(O)C)CH where | C₆ to C₂₀ | 5 |
| | alkyl | R = C₆ to C₂₀ alkyl | alkyl | |
| 30d | C₁₂H₂₅ | O(O)CCH₂(RO(O)C)CH where | C₆ to C₂₀ | 6 |
| | alkyl | R = C₆ to C₂₀ alkyl | alkyl | |
| 31d | C₁₂H₂₅ | O(O)CCH₂(RO(O)C)CH where | C₆ to C₂₀ | 7 |
| | alkyl | R = C₆ to C₂₀ alkyl | alkyl | |
| 32d | C₁₂H₂₅ | O(O)CCH₂(RO(O)C)CH where | C₆ to C₂₀ | 8 |
| | alkyl | R = C₆ to C₂₀ alkyl | alkyl | |
| 33d | C₁₂H₂₅ | O(O)CCH₂(RO(O)C)CH where | C₆ to C₂₀ | 9 |
| | alkyl | R = C₆ to C₂₀ alkyl | alkyl | |
| 34d | C₁₂H₂₅ | O(O)CCH₂(RO(O)C)CH where | C₆ to C₂₀ | 10 |
| | alkyl | R = C₆ to C₂₀ alkyl | alkyl | |
| 35d | C₁₂H₂₅ | O(O)CCH₂(RO(O)C)CH where | C₆ to C₂₀ | 11 |
| | alkyl | R = C₆ to C₂₀ alkyl | alkyl | |
| 36d | C₁₂H₂₅ | O(O)CCH₂(RO(O)C)CH where | C₆ to C₂₀ | 12 |
| | alkyl | R = C₆ to C₂₀ alkyl | alkyl | |

As stated at the outset, the compounds of general formula (1) are particularly suitable as components of detergent or cleaning agents, in particular liquid detergent or cleaning agents, due to their physical properties. Corresponding detergent or cleaning agents containing a compound of general formula (1) are therefore further subjects of this application.

A washing agent is a composition which is suitable for cleaning or caring for textiles and contains at least one textile-cleaning or textile-care active substance.

Examples of textile-cleaning active substances are
detergents, in particular nonionic or anionic detergents,
builders, such as phosphonates or water-softening polymers,

Examples of textile-care active substances are
fragrances, in particular encapsulated fragrances
fabric softeners, in particular cationic detergents and cationic polymers
enzymes, in particular proteases, amylases, lipases and cellulases
dyes, in particular textile coloring agents.

The detergents are preferably free of fluorine-containing hydrocarbon solvents.

A further subject of the application is a process for washing textiles, in the course of which a compound of the general formula (1) or a detergent containing a compound of the general formula (1) is introduced into a washing liquor. The washing liquor is preferably aqueous and free of fluorine-containing hydrocarbon solvents.

A cleaning agent is a composition which is suitable for cleaning and caring for hard surfaces and contains at least one surface-cleaning or surface-care active substance.

Examples of surface-cleaning active substances are
detergents, in particular nonionic or anionic detergents,
builders, such as phosphonates or water-softening polymers,

Examples of surface-care active substances are
corrosion inhibitors, in particular zinc salts
water repellents, in particular polymer water repellents.

The cleaning agents are preferably free of fluorine-containing hydrocarbon solvents.

A process for cleaning surfaces, in the course of which a compound of general formula (1) or a cleaning agent containing a compound of general formula (1) is introduced into a cleaning liquor, is an additional subject of this application. The cleaning liquor is preferably aqueous and free of fluorine-containing hydrocarbon solvents.

The composition of some preferred detergents or cleaning agents can be derived from the following tables (amounts given in wt. % based on the total weight of the preparation, unless otherwise indicated). The listed detergent or cleaning agents are free of fluorine-containing hydrocarbon solvents.

| | Formula 1 | Formula 2 | Formula 3 | Formula 4 |
|---|---|---|---|---|
| A compound of general formula (1) | 5 to 80 | 10 to 70 | 15 to 65 | 20 to 60 |
| Non-ionic surfactant | 5 to 50 | 10 to 45 | 15 to 40 | 20 to 35 |
| Misc. | up to 100 | up to 100 | up to 100 | up to 100 |
| | Formula 6 | Formula 2 | Formula 8 | Formula 9 |
| A compound of general formula (1) | 5 to 80 | 10 to 70 | 15 to 65 | 20 to 60 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Misc. | up to 100 | up to 100 | up to 100 | up to 100 |
| | Formula 11 | Formula 12 | Formula 13 | Formula 14 |
| A compound of general formula (1) | 5 to 80 | 10 to 70 | 15 to 65 | 20 to 60 |
| Non-ionic surfactant | 5 to 50 | 10 to 45 | 15 to 40 | 20 to 35 |
| Active washing or active cleaning | 0.1 to 14 | 0.2 to 12 | 0.5 to 10 | 0.5 to 10 |
| polymer | | | | |
| Misc. | up to 100 | up to 100 | up to 100 | up to 100 |
| | Formula 16 | Formula 17 | Formula 18 | Formula 19 |
| A compound of general formula (1) | 5 to 80 | 10 to 70 | 15 to 65 | 20 to 60 |
| Non-ionic surfactant | 5 to 50 | 10 to 45 | 15 to 40 | 20 to 35 |
| Phosphonate | 0.5 to 10 | 1 to 8 | 1 to 8 | 3 to 6 |
| Misc. | up to 100 | up to 100 | up to 100 | up to 100 |

Compounds of general formula (1) can be prepared by neutralization of a corresponding sulfonic acid with the corresponding amine. A process for preparing a compound of general formula (1) according to any of points 1 to 12, comprising the steps of
1) providing a sulfonic acid (2) of general formula R—X—SO₃H, wherein
   i) R is selected from the group of C₆ to C₂₀ alkyl groups;
   ii) X is selected from the group
      a) C₆H₄
      b) (OC₂H₄)ₙO where n=2 to 8
      c) C(O)—N(CH₃)CH₂CH₂
      d) O(O)CCH₂(RO(O)C)CH where R=C₆ to C₂₀ alkyl
2) providing an amine (3) of general formula

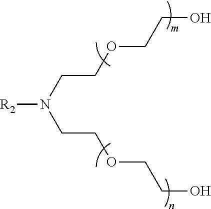

where
   i) R₂ is selected from the group of C₆ to C₂₀ alkyl groups
   ii) m+n=5 to 12.
3) mixing the anionic detergent (2) and the amine (3) to form an ionic compound of general formula (1).
   is another subject of this application.

Compounds of general formula (1) can, as described above, be prepared by reacting a sulfonic acid (2) with a corresponding tertiary amine (3). Preferred detergents based on compounds of general formula (1) can thus be described in other words by their content of sulfonic acid (2) and tertiary amine (3). The composition of some preferred detergents or cleaning agents can be derived from the following tables (amounts given in wt. % based on the total weight of the preparation, unless otherwise indicated). The listed detergent or cleaning agents are free of fluorine-containing hydrocarbon solvents.

| | Formula 21 | Formula 22 | Formula 23 | Formula 24 |
|---|---|---|---|---|
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Misc. | up to 100 | up to 100 | up to 100 | up to 100 |
| | Formula 25 | Formula 26 | Formula 27 | Formula 28 |
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Misc. | up to 100 | up to 100 | up to 100 | up to 100 |
| | Formula 31 | Formula 32 | Formula 33 | Formula 34 |
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Active washing or active cleaning | 0.1 to 14 | 0.2 to 12 | 0.5 to 10 | 0.5 to 10 |
| polymer | | | | |
| Misc. | up to 100 | up to 100 | up to 100 | up to 100 |
| | Formula 35 | Formula 36 | Formula 37 | Formula 38 |
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Phosphonate | 0.5 to 10 | 1 to 8 | 1 to 8 | 3 to 6 |
| Misc. | up to 100 | up to 100 | up to 100 | up to 100 |
| | Formula 41 | Formula 42 | Formula 43 | Formula 44 |
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Fatty acid | 1 to 12 | 1 to 12 | 2 to 10 | 2 to 10 |
| Misc. | up to 100 | up to 100 | up to 100 | up to 100 |
| | Formula 45 | Formula 46 | Formula 47 | Formula 48 |
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Water | <10 | <10 | <7 | <5 |
| Misc. | up to 100 | up to 100 | up to 100 | up to 100 |
| | Formula 51 | Formula 52 | Formula 53 | Formula 54 |
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Water | <10 | <10 | <7 | <5 |
| Misc. | up to 100 | up to 100 | up to 100 | up to 100 |
| | Formula 55 | Formula 56 | Formula 57 | Formula 58 |
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Organic solvent | 3 to 24 | 6 to 22 | 6 to 22 | 10 to 20 |
| Water | <10 | <10 | <7 | <5 |
| | Formula 61 | Formula 62 | Formula 63 | Formula 64 |
| R-X-SO₃H (2)¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Active washing or active | 0.1 to 14 | 0.2 to 12 | 0.5 to 10 | 0.5 to 10 |
| cleaning polymer | | | | |
| Organic solvent | 3 to 24 | 6 to 22 | 6 to 22 | 10 to 20 |
| Water | <10 | <10 | <7 | <5 |
| Misc. | up to 100 | up to 100 | up to 100 | up to 100 |

¹X is selected from the group
a) C₆H₄
b) (OC2H₄)nO where n = 1 to 8
c) C(O)—N(CH₃)CH₂CH₂
d) O(O)CCH₂(RO(O)C)CH where R = C₆ to C₂₀ alkyl; preferably from C₆H₄

The compounds of general formula (1) are particularly suitable for the preparation of detergent compositions with a high detergent content. In addition to their high content of sulfonic acid (2) neutralized with tertiary amine (3), these compositions are also characterized in particular by a low solvent content. The composition of some preferred highly-concentrated detergent or cleaning agent product forms can be derived from the following tables (amounts given in wt. % based on the total weight of the preparation, unless otherwise indicated). The listed detergent or cleaning agents are free of fluorine-containing hydrocarbon solvents.

| | Formula 71 | Formula 72 | Formula 73 | Formula 74 |
|---|---|---|---|---|
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Misc. | up to 100 | up to 100 | up to 100 | up to 100 |
| | Formula 75 | Formula 76 | Formula 77 | Formula 78 |
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Enzyme | 0.2 to 12 | 0.5 to 10 | 0.5 to 10 | 1 to 8 |
| preparation | | | | |
| Misc. | up to 100 | up to 100 | up to 100 | up to 100 |
| | Formula 81 | Formula 82 | Formula 83 | Formula 84 |
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Active washing | 0.1 to 14 | 0.2 to 12 | 0.5 to 10 | 0.5 to 10 |
| or active | | | | |
| cleaning polymer | | | | |
| Misc. | up to 100 | up to 100 | up to 100 | up to 100 |
| | Formula 85 | Formula 86 | Formula 87 | Formula 88 |
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Phosphonate | 0.5 to 10 | 1 to 8 | 1 to 8 | 3 to 6 |
| Misc. | up to 100 | up to 100 | up to 100 | up to 100 |
| | Formula 91 | Formula 92 | Formula 93 | Formula 94 |
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Fatty acid | 1 to 12 | 1 to 12 | 2 to 10 | 2 to 10 |
| Misc. | up to 100 | up to 100 | up to 100 | up to 100 |
| | Formula 95 | Formula 96 | Formula 97 | Formula 98 |
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Enzyme | 0.2 to 12 | 0.5 to 10 | 0.5 to 10 | 1 to 8 |
| preparation | | | | |
| Water | <5 | <5 | <3 | <3 |
| Misc. | up to 100 | up to 100 | up to 100 | up to 100 |
| | Formula | Formula | Formula | Formula |
| | 101 | 102 | 103 | 104 |
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Enzyme | 0.2 to 12 | 0.5 to 10 | 0.5 to 10 | 1 to 8 |
| preparation | | | | |
| Fatty acid | 1 to 12 | 1 to 12 | 2 to 10 | 2 to 10 |
| Water | <5 | <5 | <3 | <3 |
| Misc. | up to 100 | up to 100 | up to 100 | up to 100 |
| | Formula | Formula | Formula | Formula |
| | 105 | 106 | 107 | 108 |
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Enzyme | 0.2 to 12 | 0.5 to 10 | 0.5 to 10 | 1 to 8 |
| preparation | | | | |
| Fatty acid | 1 to 12 | 1 to 12 | 2 to 10 | 2 to 10 |
| Organic solvent | <5 | <5 | <3 | <3 |
| Water | <5 | <5 | <3 | <3 |
| Misc. | up to 100 | up to 100 | up to 100 | up to 100 |
| | Formula | Formula | Formula | Formula |
| | 111 | 112 | 113 | 114 |
| R-X-SO₃H (2)² | 20 to 40 | 20 to 40 | 25 to 35 | 25 to 35 |
| Amine (3) | 25 to 50 | 30 to 50 | 35 to 50 | 35 to 45 |
| Non-ionic | 0 to 15 | 0 to 15 | 2 to 12 | 4 to 10 |
| surfactant | | | | |
| Enzyme | 0.2 to 12 | 0.5 to 10 | 0.5 to 10 | 1 to 8 |
| preparation | | | | |
| Fatty acid | 1 to 12 | 1 to 12 | 2 to 10 | 2 to 10 |
| Active washing | 0.1 to 14 | 0.2 to 12 | 0.5 to 10 | 0.5 to 10 |
| or active | | | | |
| cleaning polymer | | | | |
| Organic solvent | <5 | <5 | <3 | <3 |
| Water | <5 | <5 | <3 | <3 |
| Misc. | up to 100 | up to 100 | up to 100 | up to 100 |

²X is selected from the group
i) C₆H₄
ii) (OC₂H₄)nO where n = 1 to 8
iii) C(O)—N(CH₃)CH₂CH₂
iv) O(O)CCH₂(RO(O)C)CH where R = C₆ to C₂₀ alkyl; preferably from C₆H₄

The detergent or cleaning agents described above are preferably free of quaternary amines and organic pigments.

This application provides, inter alia, the following subjects:

1. A compound of general formula (1)

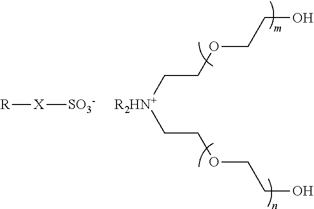

i) R is selected from the group of C₆ to C₂₀ alkyl groups;
ii) X is selected from the group
   e) C₆H₄
   f) (OC₂H₄)ₙO where n=1 to 8
   g) C(O)—N(CH₃)CH₂CH₂
   h) O(O)CCH₂(RO(O)C)CH where R=C₆ to C₂₀ alkyl
iii) R₂ is selected from the group of C₆ to C₂₀ alkyl groups
iv) where m+n=5 to 12.

2. The compound of general formula (1) according to point 1, wherein
i) R is selected from the group of C₁₀ to C₁₄ alkyl groups;
ii) X is selected from group C₆H₄
iii) R₂ is selected from the group of C₆ to C₂₀ alkyl groups, preferably C₁₀ to C₁₆ alkyl groups
iv) where m+n=5 to 12.

3. The compound of general formula (1) according to point 2, wherein
i) R is selected from the group of linear C₁₀ to C₁₄ alkyl groups.

4. The compound of general formula (1) according to one of points 2 or 3, wherein
v) R is selected from the group of secondary C₁₀ to C₁₄ alkyl groups.

5. The compounds of general formula (1) according to one of points 2 to 4, wherein
i) R is selected from the group of linear secondary C₁₀ to C₁₄ alkyl groups.

6. The compound of general formula (1) according to one of points 2 to 5, wherein
i) R represents a linear C₁₂—H₂₅ alkyl group.

7. A compound of general formula (1) according to any of points 2 to 6, wherein
i) R represents a linear secondary C₁₂H₂₅ alkyl group.

8. The compound of general formula (1) according to point 1, wherein
i) R is selected from the group of C₁₂ to C₂₀ alkyl groups;
ii) X is selected from the group selected from the group (OC₂H₄)ₙO where n=1 to 8;
iii) R₂ is selected from the group of C₆ to C₂₀ alkyl groups;
iv) where m+n=5 to 12.

9. The compound of general formula (1) according to point 1, wherein
i) R is selected from the group of C₆ to C₂₀ alkyl groups;
ii) X is selected from the group C(O)—N(CH₃)CH₂CH₂;
iii) R₂ is selected from the group of C₆ to C₂₀ alkyl groups;
iv) where m+n=5 to 12.

10. The compound of general formula (1) according to point 1, wherein
i) R is selected from the group of C₇ to C₁₇ alkyl groups;
ii) X is selected from the group O(O)CCH₂ (RO(O)C)CH where R=C₆ to C₁₇ alkyl;
iii) R₂ is selected from the group of C₆ to C₂₀ alkyl groups;
iv) where m+n=5 to 12

11. The compound of general formula (1) according to one of points 1 to 10,
iv) where m+n=5 or 6

12. The compound of general formula (1) according to point 1, wherein
i) R represents a linear secondary C₁₂H₂₅ alkyl group;
ii) X is selected from group C₆H₄
iii) R₂ is selected from the group of C₁₂H₂₅ alkyl groups
iv) m+n=5 or 6

13. A detergent or cleaning agent containing a compound of general formula (1) according to one of the preceding points.

14. A detergent or cleaning agent according to point 13, wherein the detergent or cleaning agent is free of fluorine-containing hydrocarbon solvents.

15. A process for washing textiles, over the course of which a compound of general formula (1) according to one of the preceding points 1 to 12 or a detergent or cleaning agent is introduced into a washing liquor according to point 13.

16. The process according to point 15, wherein the washing liquor is free of fluorine-containing hydrocarbon solvents.

17. The process according to any of points 15 or 16, wherein the washing liquor is aqueous.

18. The process for cleaning surfaces, over the course of which a compound of the general formula (1) according to one of the preceding points 1 to 12 is introduced into a cleaning liquor.

19. The process according to point 18, wherein the cleaning liquor is free of fluorine-containing hydrocarbon solvents.

20. The process according to one of points 18 or 19, wherein the cleaning liquor is aqueous.

21. A process for preparing a compound of general formula (1) according to any of items 1 to 12, comprising the steps of
1) providing a sulfonic acid (2) of general formula R—X—SO₃H, wherein
   i) R is selected from the group of C₆ to C₂₀ alkyl groups;
   ii) X is selected from the group
      a) C₆H₄
      b) (OC₂H₄)ₙO where n=2 to 8
      c) C(O)—N(CH₃)CH₂CH₂
      d) O(O)CCH₂(RO(O)C)CH where R=C₆ to C₂₀ alkyl
2) providing an amine (3) of general formula

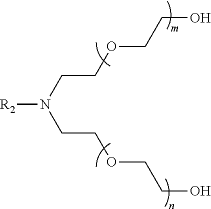

where
iii) R₂ is selected from the group of C₆ to C₂₀ alkyl groups
iv) m+n=5 to 12.

Mixing the anionic detergent (2) and the amine (3) to form an ionic compound of general formula (1).

### EXAMPLES

The following detergents were provided:

TABLE 1
Detergent compositions (wt. %)

| | V1 | V2 | V3 |
|---|---|---|---|
| 1,2-propanediol | 6.2 | 5.0 | 1.0 |
| Glycerol | 9.0 | 5.0 | — |
| Ethanol | 3.0 | 2.0 | 1.0 |
| Monoethanolamine | 2.0 | 1.0 | — |
| C₁₂₋₁₈ fatty alcohol ethoxylate, 8 EO | 25 | 25 | — |
| C₁₀₋₁₃ alkylbenzene sulfonic acid | 16 | 16 | 30 |
| Amine (3)1 | 15 | 15 | 42 |
| C₁₂₋₁₈ fatty acid | 2.0 | 7.0 | — |
| Ethoxylated polyethyleneimine | 6.0 | 5.0 | 6.0 |
| DTPMP | 0.7 | 0.7 | 0.7 |
| Enzyme preparation | 4.0 | 5.0 | 6.0 |
| polyalkoxylated alkanolamine | | 5.0 | 7.0 |
| Water, misc. | up to 100 | up to 100 | up to 100 |

1Amine (3) where R₂ selected from the group of C₁₂ to C₁₈ alkyl groups and m + n = 5

The detergent compositions are characterized by above-average washing performance and a viscous-pasty consistency advantageous for processing and dosing.

1. A compound of general formula (1) 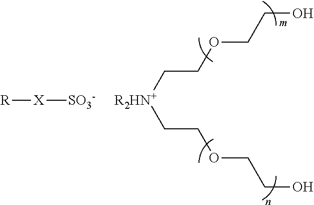
wherein
i) R is selected from the group of C₆ to C₂₀ alkyl groups;
ii) X is selected from the group
a) C₆H₄,
b) (OC₂H₄)ₙO where n=1 to 8,
c) C(O)—N(CH₃)CH₂CH₂, and
d) O(O)CCH₂(RO(O)C)CH where R=C₆ to C₂₀ alkyl;
iii) R₂ is selected from the group of C₆ to C₂₀ alkyl groups; and
iv) where m+n=5 to 12.

2. The compound of general formula (1) according to claim 1, wherein
i) R is selected from the group of C₁₀ to C₁₄ alkyl groups;
ii) X is selected from group C₆H₄;
iii) R₂ is selected from the group of C₆ to C₂₀ alkyl groups, preferably C₁₀ to C₁₆ alkyl groups; and
iv) where m+n=5 to 12.

3. The compound of general formula (1) according to claim 1, wherein
i) R represents a linear secondary C₁₂H₂₅ alkyl group;
ii) X is selected from group C₆H₄;
iii) R₂ is selected from the group of C₁₂H₂₅ alkyl groups; and
iv) m+n=5 or 6.

4. The compound of general formula (1) according to claim 1, wherein
i) R is selected from the group of C₁₂ to C₂₀ alkyl groups;
ii) X is selected from the group selected from the group (OC₂H₄)ₙO where n=1 to 8;
iii) R₂ is selected from the group of C₆ to C₂₀ alkyl groups; and
iv) where m+n=5 to 12.

5. The compound of general formula (1) according to claim 1, wherein
i) R is selected from the group of C₆ to C₂₀ alkyl groups;
ii) X is selected from the group C(O)—N(CH₃)CH₂CH₂;
iii) R₂ is selected from the group of C₆ to C₂₀ alkyl groups; and
iv) where m+n=5 to 12.

6. The compound of general formula (1) according to claim 1, wherein
i) R is selected from the group of C₇ to C₁₇ alkyl groups;
ii) X is selected from the group O(O)CCH₂(RO(O)C)CH where R=C₆ to C₁₇ alkyl;
iii) R₂ is selected from the group of C₆ to C₂₀ alkyl groups; and
iv) where m+n=5 to 12.

7. The compound of general formula (1) according to claim 1,
i) where m+n=5 or 6.

8. A detergent or cleaning agent containing a compound of general formula (1) according to claim 1.

9. A process for washing textiles, over the course of which a compound of general formula (1) according to claim 1 is introduced into a washing liquor.

10. A process for preparing a compound of the general formula (1) according to claim 1, comprising the steps of
1) providing a sulfonic acid (2) of general formula R—X—SO₃H, wherein
i) R is selected from the group of C₆ to C₂₀ alkyl groups;
ii) X is selected from the group
a) C₆H₄,
b) (OC₂H₄)ₙO where n=2 to 8,
c) C(O)—N(CH₃)CH₂CH₂, and
d) O(O)CCH₂ (RO(O)C)CH where R=C₆ to C₂₀ alkyl;
2) providing an amine (3) of general formula 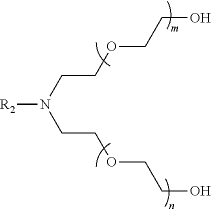
where
iii) R₂ is selected from the group of C₆ to C₂₀ alkyl groups; and
iv) m+n=5 to 12; and
3) mixing the anionic detergent (2) and the amine (3) to form an ionic compound of general formula (1).

11. The compound of general formula (1) according to claim 2, wherein
i) R represents a linear secondary C₁₂H₂₅ alkyl group;
ii) X is selected from group C₆H₄;
iii) R₂ is selected from the group of C₁₂H₂₅ alkyl groups; and
iv) m+n=5 or 6.

12. A process for washing textiles, over the course of which a detergent or cleaning agent according to claim 8 is introduced into a washing liquor.
